(19) United States
(12) Patent Application Publication
(10) Pub. No.: US 2006/0217351 A1
(43) Pub. Date: Sep. 28, 2006

(54) Method for treating cardiovascular diseases

(76) Inventors: John Thomas Brandt, Indianapolis, IN (US); Nagy Alphonse Farid, Lebanon, IN (US); Joseph Anthony Jakubowski, Indianapolis, IN (US); Kenneth John Winters, Germantown, TN (US)

Correspondence Address: ELI LILLY & COMPANY, PATENT DIVISION, P.O. BOX 6288, INDIANAPOLIS, IN, 46206-6288, US

(21) Appl. No.: 10/553,763

(22) PCT Filed: Apr. 26, 2004
(86) PCT No.: PCT/US04/11257

§ 371 Date: Oct. 21, 2005

Related U.S. Application Data
(60) Provisional application No. 60/467,903, filed on May 5, 2003.

(51) Int. Cl. A61K 31/4743; A61K 31/60; A61H 1/00

(52) U.S. Cl. 514/165; 514/301; 601/1

(57) ABSTRACT

A method of treating Cardiovascular Diseases comprising administering of a compound of formula (I) or a pharmaceutically acceptable salt, solvate, prodrug, active metabolite, racemate or enantiomer thereof, in conjunction with coronary and non-coronary intervention procedures is disclosed.

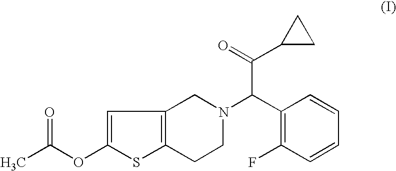

### FIELD OF THE INVENTION

This invention relates to a novel method for the treatment of Cadiovascular Disease.

### BACKGROUND OF THE INVENTION

Cardiovascular Disease is a leading cause of death and disability among most of the world's population. While the processes causing Cardiovasular Disease(s) are complex and not completely understood, an underlying etiology common to the numerous theories includes atherosclerosis due to atherosclerotic lesion formation. Artherosclerosis or artherosclerotic lesion formation has been associated with an increase in serum cholesterol, and the accumulation of cholesterol and cholesterol esters, smooth muscle cells, leukocytes, extracellular matrix and platelets in the arterial wall.

A similar etiology is also implicated in restenosis, the so-called recurrence of stenosis or arterial stricture after corrective surgery or percutaneous coronary intervention (PCI) procedures. Restenosis has been described as an accelerated atherosclerosis induced by injury (Forrester, J. S. et al., JACC, 17(3):758-769 (1991)).

Restenosis has been observed to occur after coronary artery bypass surgery, heart transplantation, atherectomy, laser ablation, and balloon angioplasty. Restenosis is most common after balloon angioplasty and other percutaneous interventions also referred to as percutaneous coronary intervention (PCI). PCI is widely used as a treatment modality in patients with coronary artery disease to reduce lumen obstruction and improve coronary blood flow. It is estimated that between 25-35% of patients develop restenosis within 1-3 months after balloon coronary angioplasty, necessitating further interventions such as repeat angioplasty or coronary bypass surgery.

The platelet inhibitor clopidogrel (marketed as Plavix® and Iscover®) has recorded some success in reducing the recurrent incidences of cardiovascular diseases caused by or exacerbated by platelet aggregation. However, clopidogrel has issues of suboptimal efficacy, non-response in certain patients, lack of tolerance in certain patients, and of being contraindicated for persons prone to bleeding. Stents, including drug-coated stents, have shown some efficacy in combating the problem of restenosis. However, stents do not treat the underlying causation of restenosis and artherosclerosis, i.e. plaque formation due to platelet activation and aggregation and/or other causes. Platelet function has also been implicated in the efficacy of non-coronary intervention procedures such as placement of stents to treat peripheral vascular disease and cerebrovascular disease. To date, there is no approved medication, treatment preventive or ameliorative agent for the cardiovascular event of restenosis adjunctive to coronary and non-coronary intervention procedures such as balloon angioplasty and stents.

It would be a significant advance in the art to discover and develop therapeutic agents that alter platelet function to avoid restenosis and other complications associated with interventional procedures to treat cardiovascular diseases. Therefore, there remains a significant need to develop more efficacious therapies that treat and/or prevent the initial and recurrent happening of cardiovascular events in patients afflicted with Cardiovascular Diseases. In particular, a significant need remains for improving or augmenting the efficiency of interventional procedures including stenting and balloon angioplasty to minimize recurrences and/or repeated interventions.

### SUMMARY OF THE INVENTION

The present invention provides a method for treating and/or preventing Cardiovascular Diseases and recurrence thereof, in a patient in need thereof, comprising the following steps performed in any order:

a) administering a compound of formula I 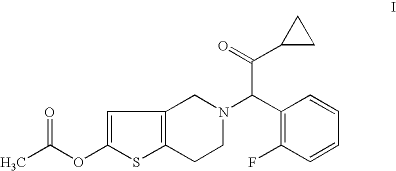
or a pharmaceutically acceptable salt, solvate, active metabolite, prodrug, racemate or enantiomer thereof; and

b) performing a PCI procedure.

The present invention also provides a method for treating and/or preventing Cardiovascular Diseases and recurrence thereof, in a patient in need thereof, comprising the following steps performed in any order:

a) administering a compound of formula I 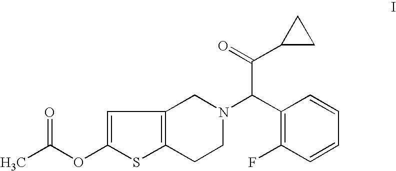
or a pharmaceutically acceptable salt, solvate, active metabolite, prodrug, racemate or enantiomer thereof in combination with aspirin; and

b) performing a PCI procedure.

The present invention also provides a method for treating and/or preventing Cardiovascular Diseases and recurrence thereof, in a patient in need thereof, comprising the following steps performed in the order described:

a) first, administering a compound of formula I 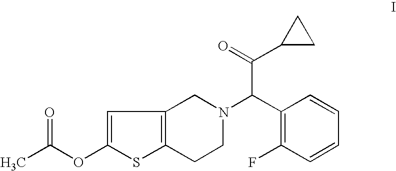
or a pharmaceutically acceptable salt, solvate, active metabolite, prodrug, racemate or enantiomer thereof, optionally in combination with aspirin;

b) second, performing a PCI procedure; and

c) third, administering a compound of formula I or a pharmaceutically acceptable salt, solvate, active metabolite, prodrug, racemate or enantiomer thereof, optionally in combination with aspirin.

The present invention also provides a method for treating and/or preventing Cardiovascular Diseases in a patient comprising in order the steps of:

a) administering a therapeutically effective amount of a compound of formula I, or a pharmaceutically acceptable salt, solvate, active metabolite, prodrug, racemate or enantiomer thereof, optionally in combination with aspirin or other cardio protective agent about 2 to 30 days prior to performing the PCI procedure

b) performing a PCI procedure, and

c) administering a therapeutically effective amount of a compound of formula I, or a pharmaceutically acceptable salt, solvate, active metabolite, prodrug, racemate or enantiomer thereof, optionally in combination with aspirin or other cardio protective agent about 0 to 365 days after performance of the PCI procedure.

The present invention also relates to the use of a therapeutically effective amount of a compound of formula I or a pharmaceutically acceptable salt, solvate, prodrug, active metabolite, racemate or enantiomer thereof, in conjunction with a stent for treating and/or preventing recurrence of Cardiovascular Diseases.

The present invention also relates to the use of a therapeutically effective amount of a compound of formula I or a pharmaceutically acceptable salt, solvate, prodrug, active metabolite, racemate or enantiomer thereof, in conjunction with a stent for treating and/or preventing recurrence of peripheral vascular disease.

The present invention also relates to the use of a therapeutically effective amount of a compound of formula I or a pharmaceutically acceptable salt, solvate, prodrug, active metabolite, racemate or enantiomer thereof, in conjunction with a stent for treating and/or preventing recurrence of cerebrovascular disease.

The present invention also provides the combination therapy of a compound of formula I or a pharmaceutically acceptable salt, solvate, prodrug, active metabolite, racemate or enantiomer thereof, in conjunction with a stent impregnated (coated) with the compound of formula I, a pharmaceutically acceptable salt, prodrug, active metabolite, racemate or enantiomer thereof, and/or other cardio-protective agent(s) for treating and/or preventing Cardiovascular Diseases and/or recurrences thereof.

The present invention relates to the manufacture of a device coated or impregnated with a compound of formula I or a pharmaceutically acceptable salt, solvate, prodrug, active metabolite, racemate or enantiomer thereof, for the treatment, prevention or amelioration of Cardiovascular Diseases.

The present invention relates to the use of 2-acetoxy-5-(α-cyclopropylcarbonyl-2-fluorobenzyl))-4,5,6,7-tetrahydrothieno[3,2-c]pyridine hydrochloric acid addition salt in combination therapy with a stent for the treatment and/or prevention of Cardiovascular Diseases.

### Definitions

The term, “Cardiovascular Diseases” refers to diseases treatable, preventable, or able to be ameliorated by performance of interventional procedures including coronary (PCI) and non-coronary interventions. Examples of cardiovascular diseases encompassed by the invention include coronary occlusion, restenosis, acute coronary syndrome (ACS), high risk vascular diseases (HRVD), cerebro vascular aneurysm (CVA), congestive heart failure, cardiac alternation, ventricular aneurysm, mural aneurysm, myocardial infarction, cardiac arrest, cardiac dysrhythmia including atrial fibrillation, cardiac edema, cardiac dyspnea, cardiac failure, tachycardia, cardiac hemoptysis, cardiac incompetence, cardiac murmur, cardiac syncope, cardiac tamponade, cerebrovascular disease and/or peripheral artery disease

“Administering” as used herein is intended to include various routes of administration, particularly oral, which allow for the compound of formula I to perform its intended function of treating and/or preventing the occurrence or recurrence of Cardiovascular Diseases as part of the combination therapy (treatment) with an interventional procedure such as a PCI procedure. Such administration by virtue of the combination treatment includes the performance of a PCI procedure e.g. the implantation of stent, or performance of balloon angioplasty.

The term “treatment” as used herein refers to the amelioration, inhibition, prevention of occurrence or recurrence, reduction in severity or effect of cardiovascular diseases including but not limited to restenosis, acute coronary syndromes, myocardial infarction, cerebro vascular aneurysm, and high risk vascular diseases by the use of a PCI or other interventional procedure in conjunction with treatment with a compound of formula I.

The term “effective amount” as used herein refers to the amount of a compound of formula I and/or other cardio protective agent (drug) necessary or sufficient to treat or prevent the particular Cadiovascular Disease in a treatment regimen comprised of a compound of formula I in conjunction with PCI or other interventional procedure as prescribed by a qualified treating physician.

The effective amount may vary depending on factors known to one of skill in the art, including for example, the optional combination of compound I with aspirin, the use of drug coated stents, mode and regimen of administration, the size of the subject, genetic or behavioral predisposition to Cardiovascular Diseases or the severity and recurrence thereof. One of skill in the art would be able to consider these and related factors to make the appropriate determination regarding effective amount.

The phrase “other cardio protective agents” as used herein refers to therapeutic agents that have been proven and approved to provide beneficial effects (treatment and/or prevention of occurrence or recurrence) to a patient afflicted with Cardiovascular Diseases. Examples of cardio-protective agents include but are not limited to aspirin, GPIIb/IIIa inhibitors, statins such as HMG-CoA reductase inhibitors, super statins, acyl CoA-cholesterol O-acyltransferase (ACAT) inhibitors, anticoagulants, thienopyridines, and other lipid modifying agents.

The phrase “Pharmaceutically acceptable carrier” refers to any substance co-administered with the compound of formula I (excluding of course the stent or other angioplasty devise) and which allows the compound to perform its intended function. Examples of such carriers include solutions, solvents, dispersion media, delay agents, emulsions, microparticles and the like for combination therapies.

The phrases “combination therapy,” “combination treatment,” “in conjunction with,” “combination of a compound of formula I and stent,” and “in conjunction with a PCI procedure” if and as used herein are synonymous and indicate that a patient who is a candidate for a PCI or other interventional procedure is administered a therapeutically effective dose(s) of a compound of formula I or a pharmaceutically acceptable salt, prodrug, active metabolite, racemate or enantiomer thereof, optionally in combination with aspirin at a reasonable period of time prior to and/or after the PCI or other interventional procedure. A reasonable period of time for administering the compound of formula I, optionally with aspirin, prior to PCI or other interventional procedure may be up to about sixty days prior and may include no prior administration. The purpose of the prior administration is to achieve on-going beneficial effect plus a rapid onset of an effect on platelet function prior to the intervention procedure, and over and above the rapid onset characteristic of a compound of formula I, particularly the HCl salt, thereby maximizing the potential benefit to the patient. The dosing of a, compound of formula I prior to an interventional procedure such as stenting or balloon angioplasty may not be practical or necessary in emergency situations. For the purpose of this invention a reasonable period after PCI or other interventional procedure, for conjunctive treatment with a compound of formula I, may be a period of from about 30 days to about 700 days, and preferably from about 30 days to about 365 days. Ultimately, the precise period of therapy according to this invention is a determination to be made by the treating or attending physician and tailored to the particular patient.

### PREFERRED EMBODIMENTS OF THE INVENTION

One embodiment of the present invention is the use of a pharmaceutical composition comprising a therapeutically effective amount of a compound of formula I, or pharmaceutically acceptable salt, solvate, racemate or enantiomer thereof, in conjunction with an interventional procedure such as PCI for the treatment and/or prevention of Cardiovascular Diseases and recurrence thereof.

Also preferred is the use the combination of aspirin and a compound of formula I,

or pharmaceutically acceptable salt, solvate, racemate or enantiomer thereof, in conjunction with a PCI procedure such as stent for the treatment and/or prevention of Cardiovascular Diseases and recurrence thereof.

Also preferred for the purpose of the invention is the use of a compound of formula I, or a pharmaceutically acceptable salt, solvate, racemate or enantiomer thereof, in conjunction with a stent procedure for the treatment and/or prevention of restenosis in peripheral and/or cerebro vascular diseases.

Also preferred for the purpose of the invention is the use of the combination of aspirin and a compound of formula I, pharmaceutically acceptable salt, solvate, racemate or enantiomer thereof, in conjunction with a stent procedure for the treatment and/or prevention of restenosis in patients afflicted with ACS, CVA or HRVD.

In a preferred embodiment, the compound of formula I is combined with aspirin or other cardio protective agent and administered in conjunction with the PCI or other interventional procedure. Preferred interventional procedure for the purpose of the invention is the placement of stent. The manufacture and use of stents are well known in the art. The object of the invention is neither the manufacture nor the use of stent but the use of a compound of formula I in conjunction with an interventional procedure such as stent for the treatment and/or prevention of Cardiovascular Diseases or recurrence thereof.

A preferred compound for the practice of the invention is 2-Acetoxy-5-α-cyclopropylcarbonyl-2-fluorobenzyl)-4,5,6,7-tetrahydrothieno[3,2-c]pyridine hydrochloride having the following formula:

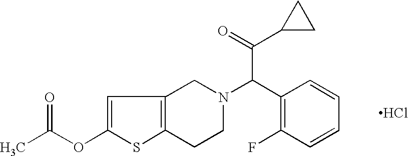

Also preferred for the practice of the invention is the compound 2-Acetoxy-5-α-cyclopropylcarbonyl-2-fluorobenzyl)-4,5,6,7-tetrahydrothieno[3,2-c]pyridine maleate, which has the following formula:

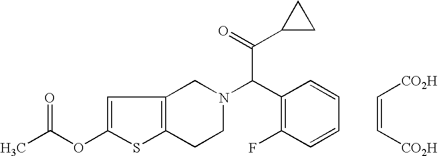

Also preferred for the practice of the invention is the active metabolite of the compound of formula I represented by the structure II:

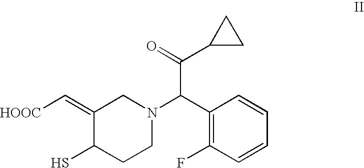

The active metabolite is a mixture of four enantiomers each of which has shown dose dependent anti-platelet aggregation ability, and thus useful for the practice of the invention. The RS enantiomer has been shown to be most potent and is therefore preferred.

Also preferred for the practice of the invention is a prodrug of the compound of formula I presented by formulae III and IV, their respective pharmaceutically acceptable salts, solvates, racemates or enantiomers thereof: 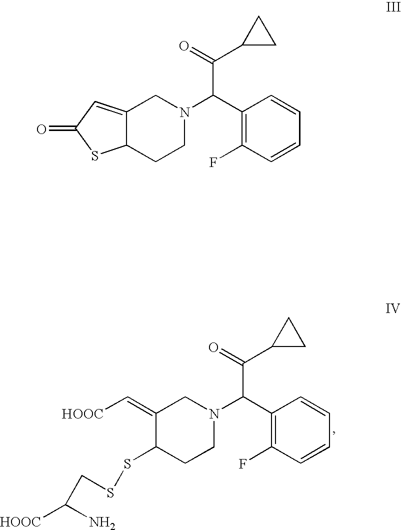
and other conjugates, derivatives or homologs of compound I or II, which may readily cleave to form the active metabolite II. Procedures and processes for making prodrugs are described herein, are known to one of skill in the art, or may be arrived at with minimal experimentation or modifications from those procedures known to one of skill in the art.

### Preparing Compounds of the Invention

A 2-acetoxy-5-(α-cyclopropylcarbonyl-2-fluorobenzyl)-4,5,6,7-tetrahydrothieno[3,2-c]pyridine or the acid addition salt has an asymmetric carbon in their molecule and in each compound two isomers having R and S configuration can exist. The present invention encompasses an individual isomer or a mixture of these isomers in optional proportions. An optically active isomer of 2-acetoxy-5-(α-cyclopropylcarbonyl-2-fluorobenzyl)-4,5,6,7-tetrahydrothieno[3,2-c]pyridine acid addition salt is prepared using an optically active starting material or is isolated from a racemic mixture of synthetically prepared 2-acetoxy-5-(α-cyclopropylcarbonyl-2-fluorobenzyl)-4,5,6,7-tetrahydrothieno[3,2-c]pyridine acid addition salt by a conventional optical resolution.

Under certain conditions when a (2-acetoxy-5-(α-cyclopropylcarbonyl-2-fluorobenzyl)-4,5,6,7-tetrahydrothieno[3,2-c]pyridine acid addition salt is allowed to stand in contact with the atmosphere or is recrystallized, it may absorb water or may take up water to form a hydrate. The present invention encompasses these hydrates.

The compound of formula I may be prepared by a variety of methods, particularly those disclosed in U.S. Pat. No. 5,288,726, the entire content of which is incorporated herein by reference. The acid addition salts of the compound of formula I may be prepared following procedures disclosed in PCT application WO 02/04461, published Jan. 17, 2002.

An acid salt of the compound of formula I (2-acetoxy-5-(α-cyclopropylcarbonyl-2-fluorobenzyl)-4,5,6,7-tetrahydrothieno[3,2-c]pyridine) is prepared in the presence or absence of an inert solvent but preferably in an inert solvent by addition of 2-acetoxy-5-(α-cyclopropylcarbonyl-2-fluorobenzyl)-4,5,6,7-tetrahydrothieno[3,2-c]pyridine, which is synthesized by a method described in U.S. Pat. No. 5,288,726, to an acid (preferably hydrochloric acid, hydrogen chloride (gas), or maleic acid; more preferably concentrated hydrochloric acid. It may also be prepared in the presence or absence of an inert solvent by dropwise addition or addition of an acid (preferably hydrochloric acid, hydrogen chloride (gas), or maleic acid; more preferably concentrated hydrochloric acid or maleic acid; most preferably concentrated hydrochloric acid) to 2-acetoxy-5-α-cyclopropylcarbonyl-2-fluorobenzyl)-4,5,6,7-tetrahydrothieno[3,2-c]pyridine. In the latter procedure, the crystalline seeds of said salt can be added, if necessary. The amount of acid (preferably hydrochloric or maleic) to be added is from is from 0.1 equivalent to 2.0 equivalent, but preferably from 0.5 to 1.5 and more preferably about 1.0 equivalent of acid. One of skill in the art is aware that the exact amount needed may be monitored during the experiment and is dependent on factors such as purity of reagents, molar equivalents of H⁺ ions per mole of acid, and the crystal lattice or lack thereof.

The solvent used in the above reaction is not particularly restricted provided that it has no adverse effect on the reaction and it can dissolve a starting material in some extent. The example of such solvent includes an aliphatic hydrocarbon such as hexane, cyclohexane, heptane, liguloin or petroleum ether; an aromatic hydrocarbon such as benzene, toluene or xylene; a halogeno-hydrocarbon such as dichloromethane, chloroform, carbon tetrachloride, 1,2-dichloroethane, chlorobenzene, or dichlorobenzene; an ether derivative such as diethyl ether, diisopropyl ether, tetrahydrofuran, dioxane, dimethoxyethane or diethylene glycol dimethyl ether; a ketone derivative such as acetone, methyl ethyl ketone, or diethyl ketone; an ester derivative such as ethyl acetate, propyl acetate, or butyl acetate: a carboxylic acid derivative such as acetic acid or propionic acid; or a nitrile derivative such as acetonitrile, or propionitrile. For preparing the hydrochloride salt, the preferable solvent is an ether derivative, a ketone derivative, an ester derivative, a carboxylic acid derivative, or a nitrile derivative, more preferable solvent is tetrahydrofuran, dioxane, acetone, methyl ethyl ketone, ethyl acetate, acetic acid or acetonitrile, and still more preferable solvent is tetrahydrofuran, dioxane, acetic acid or acetone. Acetone is most preferred. On the other hand, for preparation of the maleate salt, the prefered solvent is an ether derivative, a ketone derivative, as ester derivative or a nitrile derivative. More preferred as solvent is tetrahydrofuran, dioxane, acetone, methyl ethyl ketone, ethyl acetate, or acetonitrile. Acetone is most preferred.

The reaction temperature will vary with reagent, solvent and the like and usually is from −20° C. to 100° C., preferably from 0° C. to 70° C. With respect to the hydrochloride salt, the reaction temperature is from 30° C. to 60° C. and preferably from 40° C. to 55° C.

With respect to preparation of the maleic acid salt, preferably reaction is carried out by addition 2-acetoxy-5-(α-cyclopropylcarbonyl-2-fluorobenzyl)-4,5,6,7-tetrahydrothieno[3,2-c]pyridine to a solution of maleic acid in acetone between 0 and 70° C. followed by allowing to stand at said temperature for 1 to 3 hours.

More preferably, the reaction is carried out by dropwise addition of one and a half of required amounts of concentrated hydrochloric acid (usually equimolar with thienopyridine derivative) to a solution of 2-acetoxy-5-(α-cyclopropylcarbonyl-2-fluorobenzyl)-4,5,6,7-tetrahydrothieno[3,2-c]pyridine in acetone between 35° C. and 60° C. (preferably between 45 and 55° C.) over from 2 minutes to 10 minutes. If necessary, crystalline seeds of said salt are added, followed by allowing to stand at said temperature for 30 minutes to 2 hours; and then by further dropwise addition of the residual half of required amounts of hydrochloride to the reaction mixture over from 30 minutes to 2 hours followed by allowing to stand at said temperature for 1 to 3 hours.

After the reaction a 2-acetoxy-5-(α-cyclopropylcarbonyl-2-fluorobenzyl)-4,5,6,7-tetrahydrothieno[3,2-c]pyridine acid addition salt is isolated from a reaction mixture by conventional methods. For example, after the reaction, resulting crystal was isolated by filtration to afford a desired product or solvent of a reaction mixture was evaporated to afford a desired product. The product, if necessary, can be purified by recrystallization, reprecipitation or chromatography.

Active metabolites are formed in-vivo but may also be prepared using procedures known to one of skill in the art or by modifications thereof as stated supra. Procedures and processes for making prodrugs are known to one of skill in the art or may be arrived at with minimal experimentation or modifications from those known to one of skill in the art. Prodrugs of the active metabolite may be formed in vivo or may be prepared by one of skill in the art using disclosed procedures for compound of formula I with variations thereof.

The manufacture, clinical preparation, method of using and/or implantation of the PCI device such as a stent or balloon angioplasty is not the object of the invention. One of skill is aware of the optimum procedures and the appropriate design and procedure for accomplishing the particular PCI procedure. The processes and procedures for the manufacture of stents including drug-coated stents have been described in U.S. Pat. No. 6,554,856, and U.S. Pat. No. 6,554,157 and references disclosed therein, the entire applicable contents of which have been incorporated herein by reference. The present invention encompasses the manufacture of a stent coated or impregnated with a compound of formula I or a pharmaceutically acceptable salt, solvate, prodrug, active metabolite, racemate or enantiomer thereof, for the treatment, prevention or amelioration of Cardiovascular Diseases. The unobviousness of a stent impregnated or coated as above is supported by the superior, unexpected and beneficial effects of a compound of formula I compared to other cardiovascular agents coated on a stent hitherto.

### METHOD OF USING THE INVENTION

The practice of the invention comprises steps preferably in the order

a) treatment of a patient in need of a PCI procedure with a therapeutically effective amount of a compound of formula I, or a pharmaceutically acceptable salt, solvate, active metabolite, prodrug, racemate or enantiomer thereof, optionally in combination with aspirin or other cardio protective agent about 2 to 30 days prior to performing the PCI procedure

b) performing the PCI procedure

c) treatment of the patient with a therapeutically effective amount of a compound of formula I, or a pharmaceutically acceptable salt, solvate, active metabolite, prodrug, racemate or enantiomer thereof, optionally in combination with aspirin or other cardio protective agent about 0 to 365 days after performance of the PCI procedure.

The combination or conjunctive use of a compound of formula I or a pharmaceutically acceptable salt, prodrug, active metabolite, racemate or enantiomer thereof, in conjunction with a stent is believed to achieve its beneficial therapeutic action by the unexpected effect of simultaneously providing stenting action (prevention of restenosis) and superior inhibition of platelet aggregation, and thereby treating and/or preventing Cardiovascular Diseases and/or recurrences thereof, more efficiently than with either the compound of formula I or stent alone.

The advantages to be obtained by the use of a compound of formula I in conjunction with a stent or other PCI procedure are buttressed by the unexpectedly superior results for the compound of formula I compared to clopidogrel.

For example, it has been shown that the compound of formula I has superior anti-platelet aggregation properties compared to clopidogrel (Plavix®). In a comparative study, the compound of formula I achieved greater than 50% inhibition of platelet aggregation in thirty (30) minutes compared to minimal aggregation achieved with clopidogrel and ticlopidine in the same time period. In addition to having a faster onset of action, the compound of formula I also exhibits a higher potency (approximately 10 times higher) than clopidogrel in vivo.

Furthermore, The effect of the compound of formula I in an arteriovenous shunt model determined in rats showed that the compound of formula I (0.12-3 mg/kg, p.o.) prevented thrombus formation in a dose dependent manner with an ED₅₀ value of 0.65 mg/kg. In contrast, the required doses for clopidogrel and ticlopidine were 7.0 mg/kg and 300 mg/kg, respectively (see Asai, F. et. al., “The in vivo pharmacology of CS-747, a novel antiplatelet agent with platelet ADP receptor antagonist properties,” British Journal of Pharamcology (2000), 129(7), 1439-1446.

Also, in an embolic cerebral infarction model, the compound of formula I reduced the total area of cerebral infarct in rats, in a dose related manner compared to clopidogrel bisulfate which was about 10 times less potent though showing similar, yet milder effect on cerebral infarcts.

Comparative studies also show that the compound of formula I is less toxic and/or safer than clopidogrel. Recently clinical research organization TIMI, published a protocol design for the study of the safety of CS-747 (a compound of the present invention) in conjunction with PCI

(see http://www.timi.org/files/slides/Designs%20of%20Ongoing%20Trials%202002.ppt.

The combination of a compound of formula I, a pharmaceutically acceptable salt, prodrug, active metabolite, racemate or enantiomer thereof, and aspirin for the purpose of the invention, may be accomplished by having individual or unit doses of the compound of formula I and aspirin or by having a combined prepackaged or pre-formulated dose of aspirin and the compound, pharmaceutically acceptable salt, solvate, prodrug, racemate, or enantiomer of compound I.

A particularly preferred aspect of the present invention relates to a method for treating Cardiovascular Diseases such as acute coronary syndrome, cerebro vascular disease, high risk vascular disease, coronary occlusion, congestive heart failure, cardiac alternation, ventricular aneurysm, mural aneurysm, myocardial infarction, cardiac arrest, cardiac dysrhythmia, cardiac edema, cardiac dyspnea, cardiac failure, tachycardia, cardiac hemoptysis, cardiac incompetence, cardiac murmur, cardiac syncope, cardiac tamponade and peripheral vascular disease by the administration of a compound of formula I with or without aspirin in conjunction with a stent.

The specific dose of the compound of formula I administered according to the present invention to obtain therapeutic or prophylactic effect will, of course, be determined by the particular circumstances of the patient, including, for example, the route of administration and the particular Cardiovascular Disease being treated. Typical doses will contain a non-toxic dosage level of from about 0.01 mg/kg to about 50 mg/kg of body weight of a compound of formula I. More preferred doses of the compound of formula I are tablets or capsules containing from 5 mg to 100 mg of Active Ingredient per dose to an average weight patient or calibrated for the patient's weight and health characteristics. The frequency of dosing and length of dosing are determinations to be made by the treating physician(s) to achieve maximum efficacy for the particular patient and circumstance.

The specific dose of aspirin in a combination with a compound of formula I or salt or prodrug thereof administered according to the present invention to obtain therapeutic or prophylactic effect will, of course, be determined by the particular circumstances of the patient. In general the amount of aspirin for the purpose of the present invention is about that generally approved for the particular patient population, e.g. from about 75 mg to about 300 mg of aspirin 1 to 3 times daily.

One preferred embodiment of the invention contemplates conjunctive treatment with a stent or other PCI procedure and a compound of formula I (with or without aspirin) wherein the compound of formula I is administered prior to and continues as prescribed for a reasonable period after the stent or other PCI procedure. The stent may be coated with cardio protective agents (drugs). Preferably, the stent may be coated with cardio protective drugs that are amenable to localized delivery at or around the site of occlusion. Examples of drugs that may be coated onto stents and used in a combination treatment with a compound of formula I include active metabolites of compound I, locally active statins, super statins, ACAT inhibitors, thienopyridines, aspirin, and IIb/IIIa inhibitors or locally active formulations or derivatives thereof. Other agents useful for stent-coating for the purpose of the invention include for example, paclitaxel, and rapamycin. Where a drug coated stent is used in combination with a compound of formula I, the dose of the coating drug preferably is a factor of a tenth to 20 times higher than a single, systemic or oral therapy of the same drug, or single dose formulation. The processes for manufacture of coated stents are known to one of skill in the art and are not the object of the present invention.

For the pharmaceutical formulations of compound I with or without aspirin or other cardio protective agents, any suitable carrier known to one of skill in the art may be used. In such a formulation, the carrier may be a solid, liquid, or mixture of a solid and a liquid. For example, the Active Ingredient may be dissolved in a suitable solvent at a concentration of about 2 to 200 mg/in L in a 4% dextrose/0.5% Na citrate aqueous solution. Solid form formulations for impregnation on the stent include powders and pastes. A solid carrier can be one or more substance, which may also act as lubricants, solubilizers, suspending agents, and pharmaceutically acceptable adhesive agents.

In powders, the carrier is a finely divided solid having the necessary binding properties in suitable proportions, which is in an admixture with the finely divided Active Ingredient. The powders will typically be sprayed on optionally followed by spray-on of annealing or sealing agents. The powders preferably contain from about 1 to about 99 weight percent of the Active Ingredient. Suitable solid carriers are magnesium carbonate, magnesium stearate, talc, sugar lactose, pectin, dextrin, starch, gelatin, tragacanth, methylcellulose, sodium carboxymethyl cellulose, pharmaceutically acceptable low melting waxes, and pharmaceutically acceptable adhesives.

The Active Ingredient may be dissolved or suspended in a pharmaceutically acceptable carrier, such as sterile water, sterile organic solvent or a mixture of both. The Active ingredient may also be dissolved in a suitable organic solvent, for instance aqueous propylene glycol. Dispersing the finely divided Active ingredient in aqueous starch or sodium carboxymethyl cellulose solution, or binder or pharmaceutically acceptable adhesive may result in other compositions. The solution or suspension is then impregnated on a stent by coating the admixture of active ingredient on the stent and allowing the solvent to evaporate slowly under vacuum until nearly all solvent or liquid is evaporated.

The following pharmaceutical formulations are illustrative only and are not intended to limit the scope of the invention in any way. “Active ingredient”, refers to a compound according to Formula (I) or a pharmaceutically acceptable salt, solvate, active metabolite, enantiomer or racemate or prodrug thereof with or without other cardio protective agent(s) which is/are to be administered to a patient in need thereof, in combination with a stent procedure.

### Slow Release Formulation 1

Hard gelatin powder is prepared using the following ingredients:

| | Quantity | |
|---|---|---|
| | (mg/capsule) | |
| Active ingredient | 50 | |
| Starch, dried | 200 | |
| Magnesium stearate | 10 | |
| Total | 260 | mg |

### Formulation 2

A solid composition of formula I is prepared using the ingredients below:

| | Quantity | |
|---|---|---|
| | (mg/tablet) | |
| Active ingredient | 5 | |
| Cellulose, microcrystalline | 400 | |
| Silicon dioxide, fumed | 10 | |
| Stearic acid | 5 | |
| Total | 420 | mg |

The components are blended and compressed to form a solid each weighing 425 mg which is then tableted or capsuled or admixed with a pharmaceutically acceptable adhesion agent.

### Formulation 3

A solid composition of formula I is prepared using the ingredients below:

| | Quantity | |
|---|---|---|
| | (mg/tablet) | |
| Active ingredient | 10 | |
| Cellulose, microcrystalline | 400 | |
| Silicon dioxide, fumed | 10 | |
| Stearic acid | 5 | |
| Total | 425 | mg |

The components are blended and compressed to form a solid each weighing 425 mg which is then tableted or capsiuled or admixed with a pharmaceutically acceptable adhesion agent.

### Formulation 4

A solid composition of formula I is prepared using the ingredients below:

| | Quantity | |
|---|---|---|
| | (mg/tablet) | |
| Active ingredient | 20 | |
| Cellulose, microcrystalline | 400 | |
| Silicon dioxide, fumed | 10 | |
| Stearic acid | 5 | |
| Total | 435 | mg |

The components are blended and compressed to form a solid each weighing 425 mg. The solid is then tableted or capsuled or admixed with a pharmaceutically acceptable adhesion agent.

### EXAMPLES

The following examples, and reference examples are intended to further illustrate the present invention and are not intended to limit the scope of this invention.

### Example 1

### 2-Acetoxy-5-(α-cyclopropylcarbonyl-2-fluorobenzyl)-4,5,6,7-tetrahydrothieno[3,2-c]pyridine hydrochloride (crystal A)

Concentrated hydrochloric acid (36%, 2.71 g) was added dropwise to a solution of 2-acetoxy-5-(α-cyclopropylcarbonyl-2-fluorobenzyl)-4,5,6,7-tetrahydrothieno[3,2-c]pyridine (10 g) obtained in Reference example 1 in acetone (150 ml) with stirring at room temperature (25° C.). A small amount of crystals of the desired product (crystal A was prepared by other procedure) was added to the solution and then the mixture was stirred for 90 minutes at the same temperature. The resulting crystals were isolated by filtration and the crystal were washed with a small amount of acetone and then dried at 50° C. under reduced pressure for 4 hours to give the title compound as white crystals (8.1 g, yield 74%) (crystal A).

mp: 133-136° C.

¹H NMR (CD₃OD) δppm: 0.92-0.99 (1H, m), 1.05-1.16 (2H, m), 1.23-1.34 (1H, m), 1.84-1.95 (1H, m), 2.26 (3H, s), 3.07-3.23 (2H, m), 3.57-4.39 (4H, m), 6.04 (1H, s), 6.45 (1H, brs), 7.37-7.57 (3H, m), 7.66-7.75 (1H, m);

Mass (CI, m/z): 374 (M⁺+1);

IR (KBr) νₘₐₓcm⁻¹: 1762, 1720.

### Example 2

### 2-Acetoxy-5-(α-cyclopropylcarbonyl-2-fluorobenzyl)-4,5,6,7-tetrahydrothieno[3,2-c]pyridine maleate

2-Acetoxy-5-(α-cyclopropylcarbonyl-2-fluorobenzyl)-4,5,6,7-tetrahydrothieno[3,2-c]pyridine from Reference example 1 (15.0 g) was added to a solution of maleic acid (4.43 g) in acetone (60 ml) and the mixture was stirred at room temperature (25° C.) for 2 hours. The resulting crystals were isolated by filtration and washed with a small amount of acetone and then dried at 50° C. under reduction pressure for 4 hours to give the title compound as white crystals (17.1 g. yield 92%).

mp: 171-172° C.;

¹H NMR (CD₃OD) δppm: 0.89-0.97 (1H, m), 102-1.09 (2H, m), 1.14-1.23 (1H, m), 1.94-2.03 (1H, m), 2.25 (3H, s), 3.00-3.09 (2H, m), 3.33-3.50 (2H, m), 3.88 (1H, d, J=14.9 Hz), 4.05 (1H, d, J=14.9 Hz), 5.70 (1H, s), 6.25 (2H, s), 6.40 (1H, s), 7.30-7.42 (2H, m), 7.45-7.52 (1H, m), 7.56-7.66 (1H, m);

Mass (CI, m/z): 374 (M⁺+1);

IR (KBr) νₘₐₓcm⁻¹: 1782, 1713.

### Example 3

### 2-Acetoxy-5-(α-cyclopropylcarbonyl-2-fluorobenzyl)-4,5,6,7-tetrahydrothieno[3,2-c]pyridine hydrochloride (crystal B1)

Concentrated hydrochloric acid (36%, 2.71 g) was added dropwise to a solution of 2-acetoxy-5-(α-cyclopropylcarbonyl-2-fluorobenzyl)-4,5,6,7-tetrahydrothieno[3,2-c]pyridine (10 g) obtained in Reference example 1 in acetone (100 ml) over 1 minute with stirring at 40° C. The reaction mixture was stirred at the same temperature for 60 minutes (crystals started to precipitate after 10 minutes of the addition of concentrated hydrochloric acid). The resulting crystals were isolated by filtration and the crystals were washed with acetone (20 ml) and then dried at 60° C. under reduced pressure for 2 hours to give the title compound as white crystals (9.72 g, yield 89%) (crystal B1) which has more excellent storage stability than crystal A.

mp: 166-174° C.

Mass (CI, m/z): 374 (M⁺+1);

IR (KBr) νₘₐₓcm⁻¹: 1758, 1690.

### Example 4

### 2-Acetoxy-5-(α-cyclopropylcarbonyl-2-fluorobenzyl)-4,5,6,7-tetrahydrothieno[3,2-c]pyridine hydrochloride (crystal B2)

Concentrated hydrochloric acid (36%, 6.78 g) was added dropwise to a solution of 2-acetoxy-5-(α-cyclopropylcarbonyl-2-fluorobenzyl-4,5,6,7-tetrahydrothieno[3,2-c]pyridine (50 g) obtained in Reference example 1 in acetone (750 ml) over 5 minute with stirring at 40° C. The crystal B1 (0.1 g) obtained in Example 3 was added to the reaction mixture as crystalline seeds and the resulting mixture was stirred at the same temperature for 60 minutes. To the resulting mixture was further added dropwise concentrated hydrochloric acid (36%, 6.10 g) over 60 minutes and the mixture was stirred at the same temperature for 120 minutes. The resulting crystals were isolated by filtration and the crystals were washed with acetone (100 ml) and then dried at 70° C. under reduced pressure for 3 hours to give the title compound as white crystals (47.8 g, yield 92%) (crystal B2) which has more excellent storage stability than crystal B1 obtained in Example 3.

mp 165-178° C.

IR (KBr) νₘₐₓcm⁻¹: 1758, 1690.

### Example 5

### 2-Acetoxy-5-(α-cyclopropylcarbonyl-2-fluorobenzyl)-4,5,6,7-tetrahydrothieno[3,2-c]pyridine maleate

2-Acetoxy-5-(α-cyclopropylcarbonyl-2-fluorobenzyl)-4,5,6,7-tetrahydrothieno[3,2-c]pyridine (3000 g) obtained in Reference example 1 was added to a solution of maleic acid (932 g) in acetone (15 l) warmed to 40° C. The mixture was stirred at room temperature for 2 hours. The resulting crystals were isolated by filtration and washed with acetone (4 l) and then dried at 60° C. under reduced pressure for 8 hours to give the title compound as white crystals (3538 g, yield 90%).

mp: 172-173° C.;

Mass (CI, m/z): 374 (M⁺+1);

IR (KBr) νₘₐₓcm⁻¹: 1782, 1713.

### Example 6

### 2-Acetoxy-5-(α-cyclopropylcarbonyl-2-fluorobenzyl)-4,5,6,7-tetrahydrothieno[3,2-c]pyridine hydrochloride (crystal B2)

concentrated hydrochloric acid (36%, 6.78 g) was added dropwise to a solution of 2-Acetoxy-5-(α-cyclopropylcarbonyl-2-fluorobenzyl)-4,5,6,7-tetrahydrothieno[3,2-c]pyridine (50 g) obtained in Reference example 1 in acetone (750 ml) over 5 minute with stirring at 55° C. The crystal B1 (0.1 g) obtained in Example 3 was added to the reaction mixture as crystalline seeds and the resulting mixture was stirred at the same temperature for 60 minutes. To the resulting mixture was further added dropwise concentrated hydrochloric acid (36%, 6.08 g) over 60 minutes and the mixture was stirred at the same temperature for 120 minutes. The resulting crystals were isolated by filtration and the crystal were washed with acetone (100 ml) and then dried at 70° C. under reduced pressure for 3 hours to give the title compound as white crystals (46.2 g, yield 89%) (crystal B2).

mp: 164-178° C.

Mass (CI, m/z): 374 (M⁺+1);

IR (KBr) νₘₐₓcm⁻¹: 1758, 1690.

### Reference example 1

### 2-Acetoxy-5-(α-cyclopropylcarbonyl-2-fluorobenzyl)-4,5,6,7-tetrahydrothieno[3,2-c]pyridine

### (a) Cyclopropyl 2-fluorobenzyl ketone

A solution of 2-fluorobenzylbromide (30 ml) in diethyl ether (30 ml) was added to a suspension of metallic magnesium (7.2 g) in anhydrous diethyl ether (60 ml) and the mixture was stirred at room temperature for 1 hour. The reaction mixture was added dropwise to solution of cyclopropyl cyanide (18.2 ml) in diethyl ether (120 ml) over 100 minutes. After stirring 30 minutes at room temperature the stirred mixture was heated under reflux for 1 hour. After the reaction the mixture was partitioned between ethyl acetate and saturated aqueous ammonium chloride solution. The ethyl acetate was washed successively with water, saturated aqueous sodium bicarbonate solution, and saturated aqueous sodium chloride solution and dries over anhydrous sodium sulfate and then evaporated under reduced pressure. The residue was purified by chromatography on a silica gel column using toluene as the eluant to afford the desired product (23 g containing solvent) as yellow liquid.

¹H NMR (CDCl₃) δppm: 0.82-0.98 (2H, m), 1.03-1.17 (2H, m), 1.92-2.06 (1H, m), 3.86 (2H, s), 7.10-7.30 (4H, m);

Mass (CI, m/z): 179 (M⁺+1).

### (b) 5-(α-Cyclopropylcarbonyl-2-fluorobenzyl)-2-oxo-2,4,5,6,7,7a-hexahydrothieno[3,2-c]pyridine

N-Bromosuccinimide (9.6 g) and benzoyl peroxide (0.5 g) was added to a solution of cyclopropyl 2-fluorobenzyl ketone from Reference example 1 (a) (8.7 g) in carbon tetrachloride (80 ml) and the mixture was heated under reflux for 6 hours. After the reaction toluene was added to the reaction mixture and the resulting solid was filtered off, the filtrate was concentrated under reduced pressure. The residue was purified by chromatography on a silica gel column using toluene as the eluant to afford α-cyclopropylcarbonyl-2-fluorobenzyl bromide (8.5 g) as a yellow oil.

2-Oxo-2,4,5,6,7,7a-hexahydrothieno[3,2-c]pyridine hydrochloride (4.8 g) which was prepared according to the method described in EP 192535 (Japanese Patent Application Publication No. Sho-61-246186), and potassium bicarbonate (7.6 g) was added to a solution of α-cyclopropylcarbonyl-2-fluorobenzyl bromide (6.0 g) obtained above in dimethylformamide (20 ml). After stirring the mixture at room temperature for 2 hours the reaction mixture was partitioned between ethyl acetate and water. The ethyl acetate layer was washed with saturated aqueous sodium chloride solution, dried over anhydrous magnesium sulfate and evaporated under reduced pressure. After purification of the residue by chromatography on a silica gel column using toluene/ethyl acetate=3/1 as the eluant, the product was crystallized from diisopropyl ether to afford the desired product (2.6 g, yield 35%) as pale brown crystals.

mp: 123-125° C.;

¹H NMR (CDCl₃) ppm: 0.75-0.96 (2H, m), 0.99-1.14 (2H, m), 1.83-2.01 (1H, m), 2.02-2.17 (1H, m), 2.25-2.45 and 2.47-2.62 (total 2H, each m), 2.85 and 3.10 (total 2H, each d, J=12.0 Hz), 3.88-4.01 and 4.03-4.16 (total 2H, each m), 4.85 and 4.89 (total 1H, each s), 6.03 and 6.06 (total 1H, each s), 7.10-7.45 (4H, m);

Mass (CI, m/z): 332 (M⁺+1), 262;

Anal Calcd. for C₁₈H₁₈FNO₂S: C, 65.23; H, 5.48; N, 4.23

Found: C, 65.09; H, 5.55; N, 4.20.

### (c) 2-Acetoxy-5-(α-cyclopropylcarbonyl-2-fluorobenzyl)-4,5,6,7-tetrahydrothieno[3,2-c]pyridine

Sodium hydride (60% dispersion in mineral oil, 0.35 g) was added to a solution of 5-(α-cyclopropylcarbonyl-2-fluorobenzyl)-2-oxo-2,4,5,6,7,7a-hexahydrothieno[3,2-c]pyridine (2.6 g) from reference example 1(b) in a mixture of dimethylformamide (10 ml) and acetic acid anhydride (5 ml) in an ice bath and the mixture was stirred at the same temperature for 30 minutes and then at room temperature for 3 hours. After the reaction the mixture was extracted with ethyl acetate and the extract was washed with saturated aqueous sodium chloride solution, dried over anhydrous sodium sulfate and concentrated under reduced pressure. After purification of the residue by chromatography on a silica gel column using toluene/ethyl acetate=3/1 as the eluant, the product was crystallized from diisopropyl ether to afford the title compound (1.88 g, yield 65%) as white crystals.

mp: 120-122° C.;

¹H NMR (CDCl₃) δppm: 0.80-0.95 (2H, m), 0.99-1.16 (2H, m), 2.27 (3H, s), 2.21%-2.34 (1H, m), 2.70-2.95 (4H, m), 3.47 (1H, d, J=15.0 Hz), 3.57 (1H, d, J=15.0 Hz), 4.83 (1H, s), 6.27 (1H, s), 7.10-7.55 (4H, m);

IR (KBr) νₘₐₓcm⁻¹: 1758, 1704;

Mass (CI, m/z): 374 (M⁺+1), 304;

Anal Calcd. for C₂₀H₂₀FNO₃S: C, 64.32; H, 5.40; N, 3.75

Found: C, 64.46; H, 5.39; N, 3.73.

1. A method for treating and/or preventing Cardiovascular Diseases and recurrence thereof, in a patient in need thereof, comprising the following steps performed in any order:
a) administering a compound of formula I 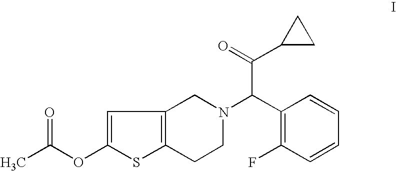
or a pharmaceutically acceptable salt, solvate, active metabolite, prodrug, racemate or enantiomer thereof; and
b) performing a PCI procedure.

2. A method for treating and/or preventing Cardiovascular Diseases and recurrence thereof, in a patient in need thereof, comprising the following steps performed in any order:
a) administering a compound of formula I 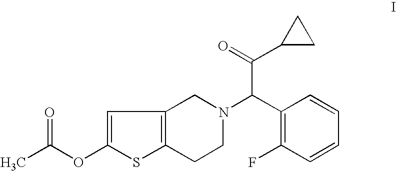
or a pharmaceutically acceptable salt, solvate, active metabolite, prodrug, racemate or enantiomer thereof in combination with aspirin; and
b) performing a PCI procedure.

3. A method for treating and/or preventing Cardiovascular Diseases and recurrence thereof, in a patient in need thereof, comprising the following steps performed in the order described:
a) first, administering a compound of formula I 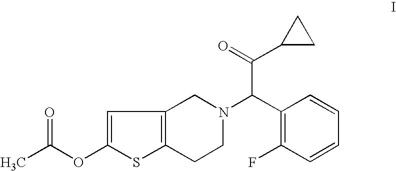
or a pharmaceutically acceptable salt, solvate, active metabolite, prodrug, racemate or enantiomer thereof, optionally in combination with aspirin;
b) second, performing a PCI procedure; and
c) third, administering a compound of formula I or a pharmaceutically acceptable salt, solvate, active metabolite, prodrug, racemate or enantiomer thereof, optionally in combination with aspirin.

4. A method according to claims 1, 2, or 3 comprising in order the steps of:
a) administering a therapeutically effective amount of a compound of formula I, or a pharmaceutically acceptable salt, solvate, active metabolite, prodrug, racemate or enantiomer thereof, optionally in combination with aspirin or other cardio protective agent about 2 to 30 days prior to performing the PCI procedure,
b) performing a PCI procedure, and
c) administering a therapeutically effective amount of a compound of formula I, or a pharmaceutically acceptable salt, solvate, active metabolite, prodrug, racemate or enantiomer thereof, optionally in combination with aspirin or other cardio protective agent about 0 to 365 days after performance of the PCI procedure.

5. A method according to claim 1, 2, or 3 wherein the compound of formula I, is 2-Acetoxy-5-(α-cyclopropylcarbonyl-2-fluorobenzyl)-4,5,6,7-tetrahydrothieno[3,2-c]pyridine hydrochloride acid addition salt or enantiomer thereof.

6. A method according to claim 1, 2 or 3 wherein the Cardiovascular Disease is selected from the group consisting of coronary occlusion, restenosis, acute coronary syndrome, high risk vascular diseases, congestive heart failure, cardiac alternation, ventricular aneurysm, mural aneurysm, myocardial infarction, cardiac arrest, cardiac dysrhythmia including atrial fibrillation, cardiac edema, cardiac dyspnea, cardiac failure, tachycardia, cardiac hemoptysis, cardiac incompetence, cardiac murmur, cardiac syncope, and cardiac tamponade.

7. A method according to claim 1, 2 or 3 wherein the Cardiovascular Disease is selected from the group consisting of ACS, CVA and HRVD.

8. A device coated or impregnated with a compound of formula I or a pharmaceutically acceptable salt, solvate, prodrug, active metabolite, racemate or enantiomer thereof, for the treatment, prevention or amelioration of Cardiovascular Diseases.

9. Use of a therapeutically effective amount of a compound of formula I or a pharmaceutically acceptable salt, solvate, prodrug, active metabolite, racemate or enantiomer thereof, in conjunction with a stent for treating and/or preventing recurrence of peripheral vascular disease.

10. Use of a therapeutically effective amount of a compound of formula I or a pharmaceutically acceptable salt, solvate, prodrug, active metabolite, racemate or enantiomer thereof, in conjunction with a stent for treating and/or preventing recurrence of cerebrovascular disease.

11. Use of a compound of formula I 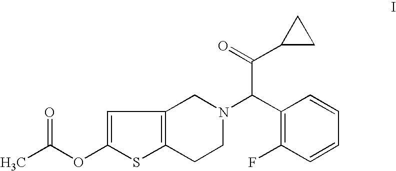
or a pharmaceutically acceptable salt, solvate, prodrug, racemate or enantiomer thereof, in conjunction with a PCI procedure for treating and/or preventing the recurrence of Cardiovascular Diseases comprising administering an effective amount of said compound of formula I optionally in combination with aspirin to a patient in need thereof.

12. A method of treating and/or preventing Cardiovascular Diseases and/or recurrences thereof, comprising administering a compound of formula I or a pharmaceutically acceptable salt, solvate, prodrug, active metabolite, racemate or enantiomer thereof, in conjunction with a stent impregnated with the compound of formula I, a pharmaceutically acceptable salt, prodrug, active metabolite, racemate or enantiomer thereof, optionally in combination with other cardio-protective agent(s) to a patient in need thereof.

13. (canceled)

14. (canceled)
